(19) United States
(12) Patent Application Publication
(10) Pub. No.: US 2024/0219348 A1
(43) Pub. Date: Jul. 4, 2024

(54) MULTI-CHANNEL DRUG DETECTION DEVICE AND METHOD

(71) Applicant: KOREA ELECTRONICS TECHNOLOGY INSTITUTE, Seongnam-si (KR)

(72) Inventors: Kunnyun KIM, Yongin-si (KR); Yeon Hwa KWAK, Seoul (KR)

(21) Appl. No.: 18/397,530

(22) Filed: Dec. 27, 2023

(30) Foreign Application Priority Data
Dec. 29, 2022 (KR) 10-2022-0189892

(51) Int. Cl. G01N 29/036; G01N 29/02; G01N 29/22; G01N 29/30; G01N 29/44

(52) U.S. Cl. CPC G01N 29/036; G01N 29/022; G01N 29/222; G01N 29/30; G01N 29/4427; G01N 29/4463; G01N 2291/0256

(57) ABSTRACT

A multi-channel drug detection method is proposed. The method may include inputting an electrical signal of a frequency included in a search range set in sensors of a sensor board, comparing magnitudes of electrical signals output by the sensors, and determining a frequency having the largest magnitude as a resonance frequency, the inputting, comparing, and determining being repeatedly performed. The method may also include determining, as a first resonance frequency, a resonance frequency determined in the inputting, comparing, and determining performed before a sample is inserted, and inserting the sample into the sensor board. The method may further include determining a second resonance frequency by repeating the inputting, comparing, and determining while moving the search range to search for a resonance frequency. The method may further include determining presence of a target material based on a difference between the first resonance frequency and the second resonance frequency.

### CROSS REFERENCE TO RELATED APPLICATION

This patent document claims the priority and benefits of Korean Patent Application No. 10-2022-0189892, filed Dec. 29, 2022, the entire contents of which are incorporated herein by reference for all purposes.

### BACKGROUND

### Technical Field

The present disclosure relates to a multi-channel drug detection device and method.

### Description of Related Technology

Various types of detection devices have been developed to verify the presence of drugs, chemicals, gases, etc. and identify types thereof.

### SUMMARY

One aspect is a multi-channel drug detection device and method that can improve a resonance frequency tracking speed.

Another aspect is a multi-channel drug detection device including a sensor board including a plurality of sensors into each of which a sample is inserted, and a main body connected to the sensor board to analyze an electrical signal provided from the sensor board, thereby determining whether a target material is preset in the sample.

In an embodiment, the sensor board may include a first connector electrically connected to the plurality of sensors, the main body may include a second connector connected to the first connector, and the first connector and the second connector may be pin array-type connectors.

In an embodiment, the multi-channel drug detection device may further include a calibration board which includes a third connector connected to the second connector and coupled to the main body to perform calibration before connecting a sensor board having a different operating frequency.

In an embodiment, the main body may include a switching unit connected to the second connector and selecting one of the plurality of sensors of the sensor board, and a matching circuit connected to the switching unit and matching impedance of a path through which an electrical signal is transmitted to one of the sensors of the sensor board to a reference value, and calibration may be performed while the calibration board is connected to the second connector.

In an embodiment, the main body may include an oscillator configured to provide electrical signals of predetermined frequencies to the plurality of sensors of the sensor board, a receiver configured to generate a signal representing a magnitude of an electrical signal obtained from the sensor, and a processor configured to control the oscillator so that the predetermined frequencies are generated and to determine whether a target material is present in the sample based on a signal obtained from the receiver, and the processor may determine an amount of the target material with reference to a reference table to correct a change in a resonance frequency of the sensors according to temperature and humidity.

In an embodiment, the processor may determine a weight according to a speed of the resonance frequency that changes immediately after the sample is inserted into the sensor board, and predict a frequency range within which the resonance frequency is to be searched for using a Kalman filter using the weight.

Another aspect is a multi-channel drug detection method including inputting an electrical signal of a frequency included in a search range set in sensors of a sensor board, comparing magnitudes of electrical signals output by the sensors, and determining a frequency having largest magnitude as a resonance frequency, the inputting, comparing, and determining being repeatedly performed at set times, determining, as a first resonance frequency, a resonance frequency determined in the inputting, comparing, and determining performed before a sample is inserted, inserting the sample into the sensor board, determining a second resonance frequency by repeating the inputting, comparing, and determining while moving the search range to search for a resonance frequency changed by a target material included in the sample, and determining presence of a target material based on a difference between the first resonance frequency and the second resonance frequency.

In an embodiment, the inputting, comparing, and determining may include setting, by a processor, a search range centered on a resonance frequency in design of the sensor board, sequentially inputting, by an oscillator, a plurality of electrical signals to the sensors within the search range, receiving, by a receiver, electrical signals output by the sensors, digitizing the received electrical signals, and providing magnitudes of the received electrical signals to the processor, and determining, by the processor, whether a peak is present when a frequency output by the oscillator and magnitude of an electrical signal received by the receiver are depicted on a two-dimensional plane, and determining a frequency corresponding to the peak as a resonance frequency.

In an embodiment, the determining a second resonance frequency may include detecting a resonance frequency changing immediately after the sample is introduced and comparing the resonance frequency with an existing resonance frequency to calculate a rate of change of the resonance frequency, selecting a weight proportional to the rate of change of the resonance frequency, predicting a frequency at which a resonance frequency is predicted to be present by inputting a frequency of a current electrical signal, a magnitude of an electrical signal provided from the receiver, and the weight to a Kalman filter, and moving a search range, within which search is to be performed in the inputting, comparing, and determining, centered on the predicted frequency, and performing the inputting, comparing, and determining according to the moved search range, performing the moving the search range again when the resonance frequency is not determined since there is no peak, and determining a resonance frequency as a second resonance frequency when the resonance frequency is determined.

In an embodiment, the determining as a first resonance frequency may include determining, as a first resonance frequency, a resonance frequency determined in the inputting, comparing, and determining performed before inserting the sample, and selecting a reference table corresponding to the first resonance frequency from among a plurality of reference tables stored in advance, and the plurality of reference tables may include a first resonance frequency value of a sensor according to temperature and humidity, and include an amount of a target material matched according to an amount of change in resonance frequency at a specific temperature and humidity.

In an embodiment, the multi-channel drug detection method may further include determining, by the processor, an amount of a target material according to an amount of change in resonance frequency with reference to a reference table including an amount of a target material matched according to an amount of change in resonance frequency at a specific temperature and humidity.

In an embodiment, the multi-channel drug detection method may further include coupling a calibration board to a main body before coupling to another sensor board having an operating frequency band different from an existing operating frequency band, inputting an electrical signal for each of a plurality of test devices of the calibration board, receiving electrical signals output by the test devices, and controlling a matching circuit so that impedance of a path through which the electrical signal is transmitted is matched to a predetermined value, and separating the calibration board from the main body.

In an embodiment, the inputting, receiving, and controlling may include inputting, by the oscillator, electrical signals of frequencies included in an operating frequency band of the sensor board to the plurality of sensors, receiving, by the receiver, electrical signals output from the plurality of sensors, digitizing the electrical signals, and providing the electrical signals to the processor, and controlling, by the processor, the matching circuit so that impedance of a path through which an electrical signal is transmitted in the main body is matched to a predetermined value based on magnitude of a signal provided from the receiver.

The features and advantages of the present disclosure will become more apparent from the following detailed description based on the accompanying drawings.

Prior to this, terms or words used in this specification and claims should not be interpreted as having usual and dictionary meaning, and should be interpreted as having meanings and concepts consistent with the technical idea of the present disclosure based on the principle that the inventor may appropriately define the concept of terms in order to describe the present disclosure in the best way.

### BRIEF DESCRIPTION OF THE DRAWINGS

The above and other objects, features and other advantages of the present invention will be more clearly understood from the following detailed description taken in conjunction with the accompanying drawings.

FIG. 1 is a diagram illustrating a multi-channel drug detection device based on an embodiment.

FIG. 2 is a diagram illustrating a sensor board and a main body based on an embodiment.

FIG. 3 is a diagram illustrating a multi-channel drug detection method based on an embodiment.

FIG. 4 is a diagram describing the sensor board based on an embodiment.

FIG. 5 is a diagram describing a Kalman filter based on an embodiment.

FIG. 6 is a diagram describing resonance frequency tracking of a comparative example and an exemplary embodiment.

FIG. 7 is a diagram illustrating a reference table based on an embodiment.

FIG. 8 is a diagram illustrating a calibration board and the main body based on an embodiment.

FIG. 9 is a diagram describing a calibration operation based on an embodiment.

### DETAILED DESCRIPTION

A detection device may use an acoustic wave sensor. The acoustic wave sensor has a receptor, which specifically binds with a target material, attached to a body made of a piezoelectric element, and may measure changes in a resonance frequency of the piezoelectric element when the target material binds with the receptor. One detection device may include a plurality of acoustic wave sensors to detect various chemicals. Detection of drugs classified as narcotics needs to be performed in the field, and the detection device needs to be portable, simple, and easy to use.

The objects, advantages, and features of the present disclosure will become more apparent from the following detailed description and preferred embodiments taken in conjunction with the accompanying drawings. However, the present disclosure is not necessarily limited thereto. Further, in describing the present disclosure, when it is determined that a detailed description of related known technology may unnecessarily obscure the gist of the present disclosure, the detailed description will be omitted.

In assigning reference numerals to components in the drawings, it should be noted that identical components are assigned the same reference numerals as much as possible even when the components are illustrated in different drawings, and similar components are assigned similar reference numerals.

Terms used to describe an implementation of the present disclosure are not intended to limit the disclosure. It should be noted that singular expressions include plural expressions unless the context clearly dictates otherwise.

The drawings may be schematic or exaggerated to illustrate implementation examples.

In this document, an expression such as “have,” “may have”, “includes,” or “may include” indicates the presence of the corresponding feature (for example, a numerical value, a function, an operation, or a component such as a part), and does not rule out the presence of additional features.

Terms such as “one”, “other”, “another”, “first”, “second”, etc. are used to distinguish one component from another, and components are not limited by the above terms.

The implementation examples described in this document and the accompanying drawings are not intended to limit the disclosure to specific embodiments. The present disclosure should be understood to include various modifications, equivalents, and/or alternatives of the implementation examples.

Hereinafter, an embodiment of the present disclosure will be described in detail with reference to the attached drawings.

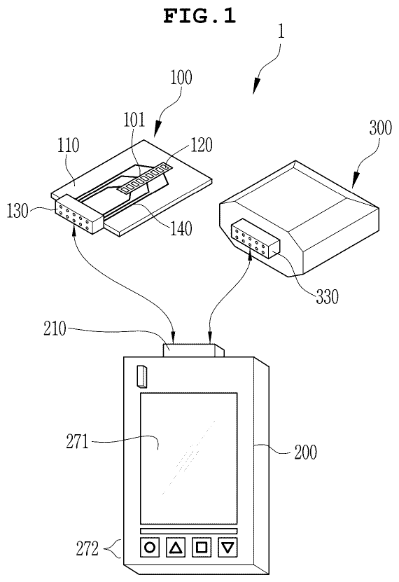

FIG. 1 is a diagram illustrating a multi-channel drug detection device 1 based on an embodiment.

The multi-channel drug detection device 1 may include a sensor board 100 including a plurality of sensors 120 into which a sample is inserted, and a main body 200 connected to the sensor board 100 to analyze an electrical signal provided from the sensor board 100, thereby determining whether a target material is present. The multi-channel drug detection device 1 may further include a calibration board 300 used for calibration.

The multi-channel drug detection device 1 based on an embodiment may be implemented as a portable device. The sample may include a liquid in which the target material may be present. The sample may be prepared by dissolving a material obtained in the field in a solvent. Alternatively, a liquid obtained in the field may be used as the sample without change. A user may detect the target material by inserting the sample into the sensor board 100 of the multi-channel drug detection device 1. A sample insertion portion 101 for insertion of a sample may be formed in the sensor board 100. The sample insertion portion 101 may be formed as an open space so that the sample and a sensor 120 are in contact. In this document, “multi-channel” means that the sensor board 100 includes a plurality of sensors 120, and each sensor 120 may detect a different target material, so that there is a plurality of channels for detecting target materials.

The user may connect the calibration board 300 to the main body 200, perform calibration, and then remove the calibration board 300. The user may connect the sensor board 100 to the main body 200 and detect drugs. The main body 200 may be connected to the calibration board 300 or the sensor board 100 through a connector. The sensor board 100 may be used for one-time use. The multi-channel drug detection device 1 may be used to detect not only drugs but also chemicals, gases, biomaterials, DNA, etc. as target materials, depending on the type of sensor 120 of the sensor board 100.

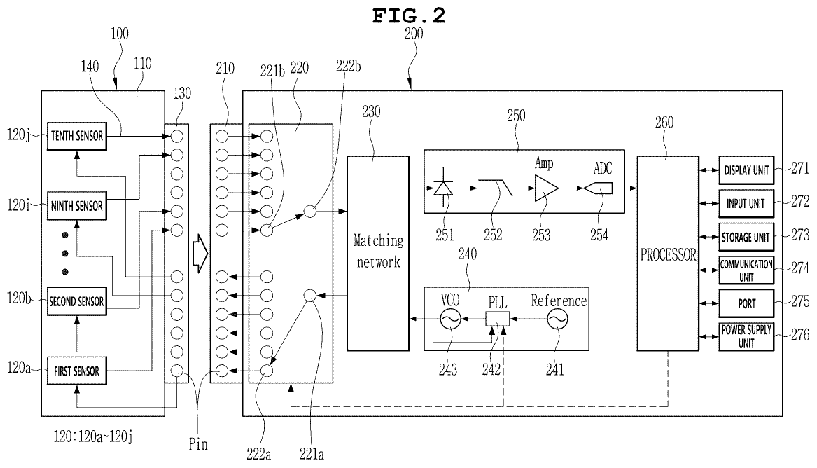

FIG. 2 is a diagram illustrating the sensor board 100 and the main body 200 based on an embodiment.

The sensor board may include a sensor substrate 110, a plurality of sensors 120, a first connector 130 for connection to the main body 200, and a sensor line 140 formed on the sensor substrate 110 to connect the first connector 130 to the plurality of sensors 120.

The sensor substrate 110 may be formed of a PCB, silicone, or various other materials. The plurality of sensors 120, the first connector 130, and the sensor line 140 may be formed on the sensor substrate 110. The sensor substrate 110 may be a body of the sensor board 100.

The sensor board 100 may include the plurality of sensors 120. Each of the plurality of sensors 120 may include a different target material. A target material refers to a material to be detected by the sensor 120. For example, a target material of a first sensor 120a may be fentanyl, a target material of a second sensor 120b may be methamphetamine, a target material of a ninth sensor 120i may be morphine, and a target material of a tenth sensor 120j may be cocaine. The number of sensors 120 may be determined depending on the number of target materials to be measured.

The sensor line 140 is a transmission line formed on the sensor substrate 110 to transmit electrical signals. The sensor line 140 may connect the first connector 130 to the plurality of sensors 120. The sensor line 140 may be designed so that the impedance is matched in order to transmit an electrical signal having a frequency of a predetermined band from the first connector 130 to the sensors 120. The sensor line 140 may be a transmission line matched to 50 Ohm.

The sensor board 100 may include the first connector 130 electrically connected to the plurality of sensors 120. The first connector 130 may be connected to a second connector 210 of the main body 200. The main body 200 may include the second connector 210 connected to the first connector 130. The first connector 130 and the second connector 210 may be pin array-type connectors.

Pin array-type connectors are less expensive and smaller than coaxial cable-type connectors. Based on an embodiment, since the sensor board 100 and the main body 200 are connected using the pin array-type connectors, the overall volume may be reduced and the price may be lowered. The pin array-type connectors may transmit electrical signals by connecting and coupling pins of the first connector 130 and pins of the second connector 210.

The main body 200 may include a switching unit 220 connected to the second connector 210 and configured to select one of the plurality of sensors 120 of the sensor board 100, a matching circuit 230 connected to the switching unit 220 and configured to match the impedance of a path through which an electrical signal is transmitted to the sensor 120 of the sensor board 100 to a reference value, an oscillator 240 configured to provide an electrical signal of a set frequency to the plurality of sensors 120 of the sensor board 100, a receiver 250 configured to generate a signal indicating the magnitude of an electrical signal obtained from the sensor 120, and a processor 260 configured to control the oscillator 240 so that a predetermined frequency is generated and to determine whether a target material is present in the sample based on a signal obtained from the receiver 250.

The switching unit 220 may output an electrical signal generated by the oscillator 240 to each pin of the second connector 210, and may receive an electrical signal output from the sensor 120 through each pin of the second connector 210 and provide the electrical signal to the receiver 250. The switching unit 220 may include a first input terminal 221a connected to the oscillator 240, a plurality of first output terminals 222a connected to the pins of the second connector 210 each connected to one end of the sensor 120, a second output terminal 222b connected to the receiver 250, and a plurality of second input terminals 221b connected to the pins of the second connector 210 each connected to the other end of the sensor 120. The switching unit 220 may perform switching so that the first input terminal 221a is connected to one of the plurality of first output terminals 222a, and may perform switching so that the second output terminal 222b is connected to one of the plurality of second input terminals 221b. For example, the switching unit 220 may perform switching to connect between the first input terminal 221a and one of the first output terminals 222a connected to one end of the first sensor 120a so that the first sensor 120a is connected to the oscillator 240, and to connect between the second output terminal 222b and one of the second input terminals 221b connected to the other end of the first sensor 120a so that the first sensor 120a is connected to the receiver 250.

The switching unit 220 may perform switching so that the first sensor 120a to an Nth sensor 120 are sequentially connected to the oscillator 240 and the receiver 250 under the control of the processor 260. With the calibration board 300 connected, the switching unit 220 may perform switching so that a first test device 320a to an Nth test device 320 are sequentially connected to the oscillator 240 and the receiver 250 under the control of the processor 260. When one sensor 120 or test device 320 is switched to be connected to the oscillator 240 and the receiver 250, the remaining sensors 120 or test devices 320 may be in a state not connected to the oscillator 240 and the receiver 250.

The matching circuit 230 may be connected between the switching unit 220 and the oscillator 240 and between the switching unit 220 and the receiver 250. The matching circuit 230 may include a resistor, an inductor, and a capacitor therein. The matching circuit 230 may also be referred to as a matching network. The matching circuit 230 may change connection and magnitude of the resistor, the inductor, and the capacitor to match the impedance of the path through which the electrical signal is transmitted to a predetermined value. For example, the impedance of the path through which the electrical signal is input to the first sensor 120a and the path through which the electrical signal output from the first sensor 120a is received may be matched to 50 Ohm.

The oscillator 240 may generate and output an electrical signal of a set frequency. The oscillator 240 may generate an electrical signal of a specific frequency under the control of the processor 260. The oscillator 240 may include a reference oscillator 241, a Phase-Locked Loop (PLL) 242, and a voltage-controlled oscillator 243 (VCO). The reference oscillator 241 may generate an electrical signal of a set frequency and output the electrical signal to the PLL 242. The PLL 242 receives feedback of a control signal input from the processor 260, an electrical signal output from the reference oscillator 241, and an output signal of the VCO 243, and outputs an output signal that controls the VCO 243. The VCO 243 may output an electrical signal of a determined frequency based on the output signal of the PLL 242. The output of the VCO 243 is the output of the oscillator 240 and may be transmitted to the switching unit 220 through the matching circuit 230.

For example, the reference oscillator 241 of the oscillator 240 may generate an electrical signal of a frequency of 10 MHz and output the electrical signal to the PLL 242, the PLL 242 may provide an output signal to the VCO 243 to reach a target frequency of a control signal input from the processor 260, and the VCO 243 may output an electrical signal according to the target frequency of the control signal. The processor 260 may perform a control operation so that the oscillator 240 outputs an electrical signal of a specific frequency by changing the target frequency of the control signal.

The receiver 250 may include a power detector 251, a filter 252, an amplifier 253, and an analog-to-digital converter 254. The receiver 250 may measure the magnitude of the electrical signal output from the sensor 120, digitize the electrical signal, and transmit the electrical signal to the processor 260. The power detector 251 may include a diode configured to rectify an electrical signal, which is an alternating current. Electrical signals passing through the power detector 251 may pass through the filter 252 to remove a signal in a high frequency band. The filter 252 may be a low-pass filter 252. The amplifier 253 may amplify the magnitude of the electrical signal passing through the filter 252. The analog-to-digital converter 254 may convert the electrical signal amplified by the amplifier 253 into a digital signal, convert the signal into a signal having the magnitude of the electrical signal, and output the signal to the processor 260. In summary, the receiver 250 may generate a signal indicating the magnitude of the electrical signal output from the sensor 120 and provide the signal to the processor 260.

The processor 260 is an element capable of performing information processing. The processor 260 may include a CPU, a GPU, an APU, and other chips capable of processing information. The main body 200 may include a plurality of processors 260.

The processor 260 may determine a frequency of an electrical signal output from the oscillator 240. The processor 260 may determine a frequency of an electrical signal to be output by the oscillator 240 using a Kalman filter using a weight. The processor 260 may provide a control signal to the oscillator 240 so that the oscillator 240 outputs an electrical signal of a predetermined frequency. The processor 260 may control the switching unit 220 so that a specific sensor 120 among the plurality of sensors 120 is connected. The processor 260 may perform a control operation so that the matching circuit 230 matches the impedance to a predetermined value. When a first resonance frequency measured before the sample is inserted into the sensor 120 and a second resonance frequency measured after the sample is inserted into the sensor 120 are different from each other, the processor 260 may determine that a target material that specifically binds with the corresponding sensor 120 is present in the sample. The processor 260 may determine the amount of the target material according to a change in resonance frequency. The processor 260 may determine the amount of the target material by referring to a reference table in order to correct changes in the resonance frequency of the sensor 120 depending on the temperature or humidity.

The processor 260 may be connected to a display unit 271, an input unit 272, a storage unit 273, a communication unit 274, and a port 275 so that communication may be performed. The processor 260 may provide an interface screen for the user to use the multi-channel drug detection device 1 through the display unit 271. The processor 260 may receive a command input by the user through the input unit 272. The processor 260 may execute program code stored in the storage unit 273 to perform a multi-channel drug detection method based on an embodiment. The processor 260 may transmit and receive data to and from the outside through the communication unit 274. The processor 260 may transmit and receive data to and from an electronic device connected to the port 275 through the port 275.

The display unit 271 may include a display for providing visual information and a speaker for providing auditory information. The display of the display unit 271 may be placed on one side of the main body 200.

The input unit 272 may receive a command input by the user. The input unit 272 may include a touch-screen, buttons, etc. The input unit 272 may be exposed to allow the user to access the main body 200.

The storage unit 273 may include a memory chip, a hard disk, a magnetic tape, etc. capable of storing data. The storage unit 273 may use a cloud storage connected through the communication unit 274. The storage unit 273 may include a sample measurement result, a set value of the matching circuit 230, a manufacturing resonance frequency value according to a type of sensor 120, a plurality of reference tables according to temperature or humidity, a Kalman filter 252 algorithm, other data for operating the multi-channel drug detection device 1, and program code written to perform each operation of the multi-channel drug detection method.

The communication unit 274 may be connected to a wired or wireless network to transmit and receive data. The communication unit 274 may use short-range wireless communication methods such as Wi-Fi and Bluetooth, mobile communication methods such as 5G, 6G, and LTE, and communication methods such as WAN, LAN, Ethernet, IPv4, and IPv6.

The port 275 may be arranged so that one end is exposed to the outside of the main body 200. The port 275 may be used to connect the main body 200 to a cable or another electronic device. It is possible to include a USB port 275, a communication port 275, and ports 275 of various other specifications.

The power supply unit 276 may supply electrical energy to the main body 200. The power supply unit 276 may supply electrical energy to the processor 260, the oscillator 240, the switching unit 220, and other components. The power supply unit 276 may include a battery, a charging circuit, and a charging terminal.

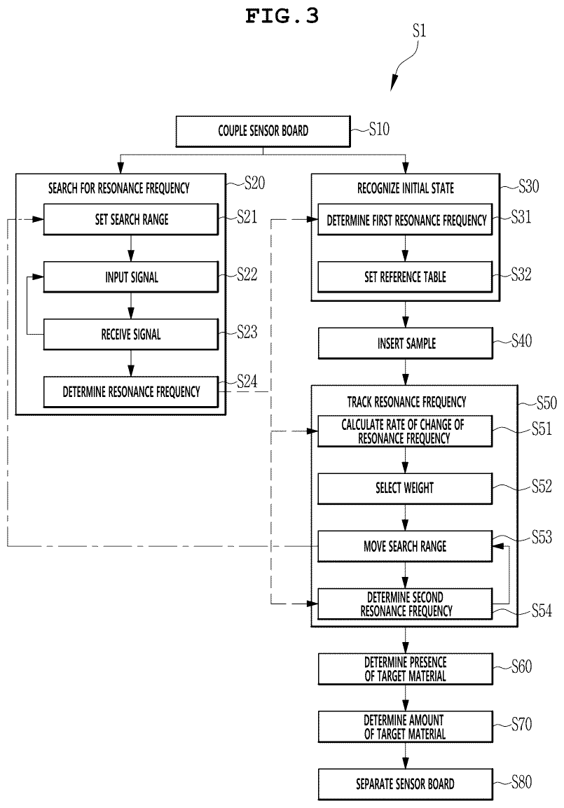

FIG. 3 is a diagram illustrating a multi-channel drug detection method based on an embodiment.

The multi-channel drug detection method based on an embodiment may include a detection operation S1. The detection operation S1 is a process of determining whether a target material is present in the sample by analyzing a resonance frequency using the sensor board 100. The detection operation S1 may include a resonance frequency search operation S20 of inputting an electrical signal of a frequency included in a search range set in the sensor 120 of the sensor board 100, comparing the magnitudes of electrical signals output by the sensor 120, and determining a frequency having the largest magnitude as a resonance frequency, an initial state recognition operation S30 of determining, as a first resonance frequency, a resonance frequency determined in the resonance frequency search operation S20 performed before inserting the sample, an operation of inserting the sample into the sensor board 100, a resonance frequency tracking operation S50 of determining a second resonance frequency by repeating the resonance frequency search operation S20 while moving the search range to search for a resonance frequency changed by a target material included in the sample, and an operation S60 of determining the presence of the target material based on a difference between the first resonance frequency and the second resonance frequency. The resonance frequency search operation S20 may be repeatedly performed at set times.

The user may perform an operation S10 of coupling the sensor board 100 to the main body 200 to perform the multi-channel drug detection method. The user may check a result of analyzing the presence and amount of the target material and then perform an operation S80 of separating the sensor board 100 from the main body 200.

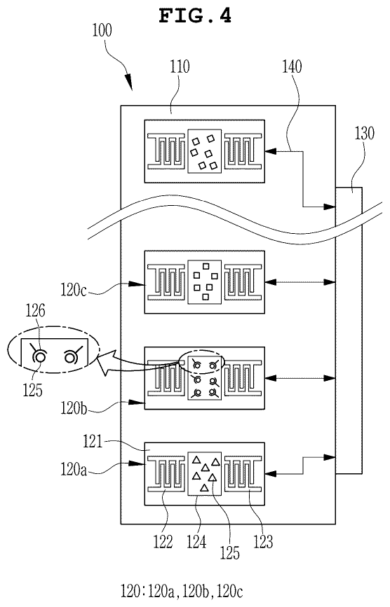

The sensor 120 disposed on the sensor board 100 of the multi-channel drug detection device 1 may be a surface acoustic wave sensor 120. To describe the multi-channel drug detection method, a structure and operation of the sensor 120 will first be described with reference to FIG. 4.

FIG. 4 is a diagram describing the sensor board 100 based on an embodiment. FIGS. 2, 3, and 4 will also be used for description.

The plurality of sensors 120 may be disposed on the sensor substrate 110. In each of the sensors 120, an input inter-digital transducer (IDT) 122 and an output IDT 123 may be formed on a piezoelectric material 121, and a detection region 124 may be formed between the input IDT 122 and the output IDT 123. A detection material 125 specifically combining with the target material may be formed in the detection region 124. When an electrical signal generated in the oscillator 240 is transmitted to the input IDT 122, the input IDT 122 generates a surface acoustic wave by vibrating the piezoelectric material 121 according to a frequency of the electrical signal, the surface acoustic wave passes through the detection region 124 and reaches the output IDT 123, and the output IDT 123 converts the surface acoustic wave transmitted through the piezoelectric material 121 into an electrical signal and outputs the converted electrical signal. In this instance, when a signal corresponding to the resonance frequency is input to the piezoelectric material 121, the magnitude of the electrical signal output by the output IDT 123 appears the largest.

When the user performs an operation of coupling the sensor board 100 to the main body 200 and inputs a measurement start command through the input unit 272, the main body 200 may repeatedly perform the resonance frequency search operation S20. The resonance frequency search operation S20 may be repeatedly performed at set times in a drug detection process. A set time interval may be significantly short. The resonance frequency determined in the resonance frequency search operation S20 may be stored in the storage unit 273 along with a determined time. The resonance frequency search operation S20 may be performed in parallel with sequentially performing the initial state recognition operation S30, the operation S40 of inserting the sample into the sensor board 100, and the resonance frequency tracking operation S50.

The resonance frequency search operation S20 may be sequentially performed for each of the plurality of sensors 120. After searching for a resonance frequency of the first sensor 120a, a resonance frequency of the second sensor 120b may be searched for, and then a resonance frequency of the Nth sensor 120 may be searched for. A process of searching for resonance frequencies from the first sensor 120a to the Nth sensor 120 and then searching for resonance frequencies from the first sensor 120a to the Nth sensor 120 may be repeated. The processor 260 may store a result of searching for the resonance frequency for each of the sensors 120 in the storage unit 273.

The resonance frequency search operation S20 may include an operation S21 in which the processor 260 sets a search range centered on a resonance frequency in the design of the sensor board 100, an operation S22 in which the oscillator 240 sequentially inputs a plurality of electrical signals to the sensors 120 within the search range, an operation S23 in which the receiver 250 receives an electrical signal output from the sensor 120, digitizes the received electrical signal, and provides the magnitude of the received electrical signal to the processor 260, and an operation S24 in which the processor 260 determines whether a peak is present when the frequency output from the oscillator 240 and the magnitude of the electrical signal received by the receiver 250 are depicted on a two-dimensional plane, and determines a frequency corresponding to the peak as the resonance frequency.

The operation S21 of setting the search range is a process of determining the search range of the frequency including a value expected to be the resonance frequency in order to search for the resonance frequency of the sensor board 100. When the sensor board 100 is connected, the processor 260 may determine a search range within a range determined based on the resonance frequency in the design of the sensor board 100. For example, when the resonance frequency in the design of the sensor 120 is 300 MHZ, the processor 260 may determine 20 MHz from 290 MHz to 310 MHz as the search range. When the search range is determined, a search interval may be determined. The search interval is a frequency interval of electrical signals output by the oscillator 240 in order within the search range. For example, when causing the oscillator 240 to output electrical signals of 292 MHZ, 294 MHZ, 296 MHZ, . . . 308 MHz, and 310 MHz, starting with an electrical signal of 290 MHz, the search interval is determined to be 2 MHZ.

In the operation S22 of inputting an electrical signal, the processor 260 controls the oscillator 240 so that a plurality of electrical signals is sequentially generated within the search range, and controls the switching unit 220 so that the sensors 120 are switched according to a determined order. The processor 260 may control the oscillator 240 so that electrical signals of a plurality of different frequencies are generated and output depending on the search range and search interval. For example, the oscillator 240 may input an electrical signal of 290 MHz to the first sensor 120a, then input an electrical signal of 292 MHZ, input an electrical signal according to the search interval, and finally input an electrical signal of 310 MHZ.

The operation S23 of receiving an electrical signal is a process of digitizing an electrical signal output from the sensor 120 to generate the magnitude of the electrical signal, and providing the magnitude to the processor 260. For example, when the processor 260 controls the oscillator 240 so that an electrical signal of 290 MHz is input to the sensor 120, the receiver 250 may generate the magnitude of the electrical signal output to the sensor 120 accordingly, and provide the magnitude to the processor 260. The operation S23 of receiving an electrical signal may be performed for each operation S22 of inputting an electrical signal. When a plurality of electrical signals is input according to the search range and search interval, there is a plurality of electrical signals output by the sensor 120, and there is a plurality of magnitudes of electrical signals received by the processor 260.

While repeating the operation S22 of inputting an electrical signal and the operation S23 of receiving an electrical signal, the processor 260 may collect the magnitude of the electrical signal output by the sensor 120 for each frequency. In the operation S24 of determining a resonance frequency, the processor 260 may compare the magnitudes of the electrical signals and determine a frequency corresponding to the peak as the resonance frequency. When frequencies input to the sensor 120 are listed on a horizontal axis in order according to the search range and the search interval, and the magnitudes of electrical signals output by the sensor 120 in response to the frequencies are depicted on a vertical axis, the peak means that a center appears convex upward. When the peak is present, a frequency at which the magnitude of the electrical signal is largest may be determined as the resonance frequency. When no peak is present, the resonance frequency cannot be determined. When frequencies input to the sensor 120 are listed on a horizontal axis in order according to the search range and the search interval, and the magnitudes of electrical signals output by the sensor 120 in response to the frequencies are depicted on a vertical axis, the case where no peak is present may mean that a shape inclined from the upper right to the lower left or a shape inclined from the upper left to the lower right appears. Such a shape appears since there is no resonance frequency within the search range, and thus the magnitude of the electrical signal output by the sensor 120 linearly appears depending on the frequency.

When a resonance frequency is determined for the first sensor 120a, the processor 260 may sequentially determine a resonance frequency for the second sensor 120b. The processor 260 may perform the operation S24 of determining a resonance frequency for each of the plurality of sensors 120.

When the user performs the operation of coupling the sensor board 100 to the main body 200 and inputs a measurement start command through the input unit 272, the main body 200 may perform the initial state recognition operation S30 simultaneously with repeatedly performing the resonance frequency search operation S20.

The initial state recognition operation S30 may include a first resonance frequency determination operation S31 in which the resonance frequency of the sensor 120 is determined in a state where no sample is input to the sensor 120. The first resonance frequency determined in the initial state recognition operation S30 reflects an influence of an environment in which the multi-channel drug detection device 1 is used. The first resonance frequency may be determined differently for each sensor 120.

Even though the sensor 120 is manufactured to have a resonance frequency by design at the time of manufacturing, there may be slight differences in the resonance frequency actually measured for each sensor 120 due to manufacturing errors. Further, since the characteristics of the piezoelectric material 121 of the sensor 120 slightly change due to temperature, humidity, etc., a difference in resonance frequency may occur.

In the initial state recognition operation S30, as the resonance frequency search operation S20 is repeatedly performed, the processor 260 may determine, as the first resonance frequency, an average value of a plurality of resonance frequencies for each sensor 120 stored in the storage unit 273 (S31). The resonance frequency may be determined differently each time the resonance frequency search operation S20 is performed due to an influence of vibration applied to the main body 200 or noise due to environmental effects. When the average value of the plurality of resonant frequencies is determined as the first resonance frequency, measurement errors caused by irregular fluctuations caused by noise may be reduced.

When the first resonance frequency is determined, the user may insert the sample into the sensor board 100. When the sample is inserted, the detection material 125 formed in the detection region 124 of the sensor 120 may be specifically bound with a target material 126 present in the sample. When the target material 126 is bound with the detection material 125, the mass of the detection region 124 may increase. For example, the first sensor 120a of FIG. 4 shows a state in which the target material 126 is not bound with the detection material 125, and the second sensor 120b of FIG. 4 shows a state in which the target material 126 is bound with the detection material 125 (see an enlarged view). When the target material 126 is bound with the detection material 125 as in the second sensor 120b of FIG. 4, a surface acoustic wave is generated by an electrical signal input to the input IDT 122. Further, since the mass of the detection region 124 changes when the surface acoustic wave passes through the detection region 124, the characteristics of the surface acoustic wave change accordingly, and the frequency of the electrical signal output by the output IDT 123 may also change due to the changed surface acoustic wave.

When the target material included in the sample is bound with the detection region 124, the resonance frequency of the sensor 120 changes. The resonance frequency tracking operation S50 is a process of tracking where the changed resonance frequency of the sensor 120 is present. After the sample is inserted into the sensor board 100, the resonance frequency determined by performing the resonance frequency tracking operation S50 is referred to as the second resonance frequency. The specific details of the resonance frequency tracking operation S50 are described later.

The case where the second resonance frequency changes compared to the first resonance frequency means that the target material 126 is bound with the detection material 125 in the detection region 124, and thus the target material 126 may be considered to be present in the sample. By performing the resonance frequency tracking operation S50 to search for the second resonance frequency, which is a resonance frequency changing after the sample is inserted, the presence or absence of the target material and the amount of the target material may be calculated by comparing the first resonance frequency with the second resonance frequency.

The processor 260 may compare the first resonance frequency with the second resonance frequency for each sensor 120 and determine that the target material is present in the sample when the second resonance frequency is lower than the first resonance frequency. For example, the first resonance frequency and the second resonance frequency of the second sensor 120b are compared, and when the second resonance frequency is lower than the first resonance frequency, it is possible to determine that a target material that specifically binds with the detection material 125 of the second sensor 120b is present in the sample.

Upon determining that the target material is present, the processor 260 may perform an operation of determining the amount of the target material. As a difference between the first resonance frequency and the second resonance frequency increases, the amount of target material may be determined to be larger. The specific details of determining the amount of the target material will be described later.

By performing the above operations, the processor 260 may detect the presence of the target material.

It takes a lot of time to perform the resonance frequency search operation S20 in all frequency ranges to search for the changed resonance frequency of the sensor 120 after the sample is inserted. Based on an embodiment, the processor 260 may predict a frequency close to the resonance frequency using a Kalman filter using a weight, thereby significantly reducing the time to search for the resonance frequency. The processor 260 may select a weight proportional to a degree to which the resonance frequency moves immediately after inserting the sample, allowing faster access to the changed resonance frequency.

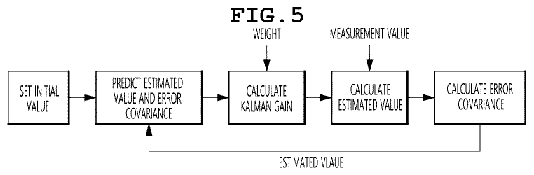

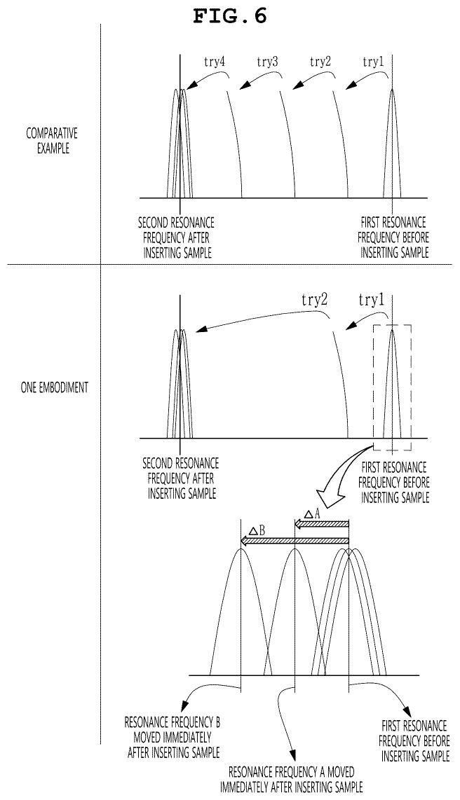

FIG. 5 is a diagram describing the Kalman filter based on an embodiment. FIG. 6 is a diagram describing resonance frequency tracking of a comparative example and an exemplary embodiment. FIG. 3 will also be used for description.

The resonance frequency tracking operation S50 may include an operation S51 of detecting the changing resonance frequency immediately after the sample is inserted and comparing the resonance frequency with the existing resonance frequency to calculate a rate of change of the resonance frequency, an operation S52 of selecting a weight proportional to the rate of change of the resonance frequency, an operation S53 of predicting a frequency at which a resonance frequency is predicted to be present by inputting a frequency of a current electrical signal, the magnitude of an electrical signal provided from the receiver 250, and a weight to the Kalman filter, and moving the search range, within which search is to be performed in the resonance frequency search operation S20, centered on the predicted frequency, and an operation S54 of performing the resonance frequency search operation S20 according to the moved search range, performing the operation S53 of moving the search range again when the resonance frequency is not determined since there is no peak, and determining a resonance frequency as the second resonance frequency when the resonance frequency is determined.

First, the Kalman filter using a weight will be described with reference to FIG. 5. The Kalman filter receives the frequency of the current electrical signal as an initial value, receives the magnitude of the electrical signal provided from the receiver 250 as a measurement value, and receives a weight. In the Kalman filter, the weight can be employed in the process of calculating the frequency at which the resonance frequency is predicted to be present, serving as an estimated value. When the weight is large, a difference between the frequency of the current electrical signal and the frequency predicted as an estimated value increases.

The operation S53 of moving the search range is a process of resetting the search range centered on a frequency predicted by inputting the frequency of the current electrical signal, the magnitude of the electrical signal provided from the receiver 250, and the weight. In other words, the search range is moved to a search range centered on a frequency predicted in a previously set search range. For example, when the search range is from 290 MHz to 310 MHz in the resonance frequency search operation S20 performed before inserting the sample, the search range moved by performing the operation S53 of moving the search range may be from 240 MHz to 260 MHz. As a result of designating the search range based on a result of predicting the frequency using the Kalman filter, the resonance frequency search operation S20 is not performed in a range from 260 MHz to 290 MHz, and thus a time required to track the resonance frequency may be reduced.

When the operation S53 of moving the search range is performed, the moved search range is set. Therefore, in the resonance frequency search operation S20 performed after the operation S53 of moving the search range, the resonance frequency is searched in the moved search range. The moved search range is different from the previous search range.

Next, the operation S54 of determining the second resonance frequency may be performed. In the operation S54 of determining the second resonance frequency, when the resonance frequency is determined as a result of performing the resonance frequency search operation S20 in the moved search range, the frequency may be determined as the second resonance frequency.

In the operation S54 of determining the second resonance frequency, when the resonance frequency search operation S20 is performed according to the moved search range, there is no peak, and thus the resonance frequency is not determined, performing the operation S53 of moving the search range again may be repeated.

FIG. 6 schematically illustrates the magnitude of the electrical signal in the search range, and illustrates a convex upward shape at the resonance frequency.

As shown in the comparative example of FIG. 6, when there is no peak in a first attempt try1 to search for the resonance frequency by moving the search range after the sample is inserted, only an inclined shape appears, and thus the resonance frequency cannot be determined, a second attempt try2 to move the search range again and search for the resonance frequency in the moved search range may be performed, and such a process may be repeated to track the resonance frequency. However, moving the search range at regular intervals, as in the comparative example, may result in a long detection time since a process of searching for the resonance frequency needs to be repeated a plurality of times.

In contrast, as shown in an exemplary embodiment of FIG. 6, in the first attempt try1 to search for the resonance frequency by moving the search range after the sample is inserted, when no peak appears and only an inclined shape appeared, and thus the resonance frequency could not be determined, it can be seen that the search range moves significantly largely due to the weight when the search range is moved again. That is, the operation S53 of moving the search range may reduce the number of times of repeating a process of receiving the weight as input of the Kalman filter, largely moving the search range according to the weight, and searching for the resonance frequency. Therefore, a detection time may be shortened.

A method of selecting a weight will be described with reference to the enlarged view of FIG. 6. In the resonance frequency tracking operation S50, the operation S51 of calculating a rate of change and the operation S52 of selecting a weight may be performed before performing the operation S53 of moving the search range.

The operation S51 of calculating a rate of change is a process of detecting the resonance frequency changing immediately after the sample is inserted, calculating a difference to the existing resonance frequency, and calculating a rate of change using a difference between a time when the resonance frequency is previously searched for and a time when the changed resonance frequency is detected. The enlarged view of FIG. 6 is a diagram enlarging and illustrating a frequency axis. Before the sample is inserted, a plurality of resonance frequencies that fluctuates due to noise near the first resonance frequency is measured. Immediately after inserting the sample, the target material and the detection material 125 included in the sample begin to bind and the resonance frequency begins to move. In the case of a resonance frequency A that moves immediately after inserting the sample, the amount of change in resonance frequency is ΔA MHz. In the case of a resonance frequency B that moves immediately after inserting the sample, the amount of change in the resonance frequency is ΔB MHz. The amount of change in the resonance frequency B is greater than that in the resonance frequency A (ΔB>ΔA). The large amount of change in resonance frequency immediately after sample is inserted may mean that the sample contains a large amount of the target material, and thus the combination of the target material and the detection material 125 is fast. Therefore, it can be expected that a difference between the second resonance frequency and the first resonance frequency to be finally searched will be large. Therefore, a larger weight may be selected when the resonance frequency B is measured than when the resonance frequency A is measured.

When a time at which the resonance frequency search operation S20 is repeated is fixed, the rate of change of the resonance frequency is proportional to the amount of change. Therefore, the rate of change may be considered to be larger when the resonance frequency B is measured than when the resonance frequency A is measured. In proportion to the rate of change, a weight when the resonance frequency B is measured may be selected to be larger than a weight when the resonance frequency A is measured.

In the operation S52 of selecting a weight, it is possible to use a method of referring to a table matching a weight value according to the rate of change of the resonance frequency. The table matching the rate of change of the resonance frequency and the weight value may be created through experimentation. When the rate of change of the resonance frequency is calculated in operation S51 of calculating the rate of change of the resonance frequency, a weight value matching the rate of change of the resonance frequency may be selected by referring to the table in the storage unit 273.

When the weight value selected through this process is input to the Kalman filter, the Kalman filter outputs a result in which a frequency at which the resonance frequency is predicted to be present moves further. Therefore, the detection time based on an exemplary embodiment may be shortened compared to the comparative example.

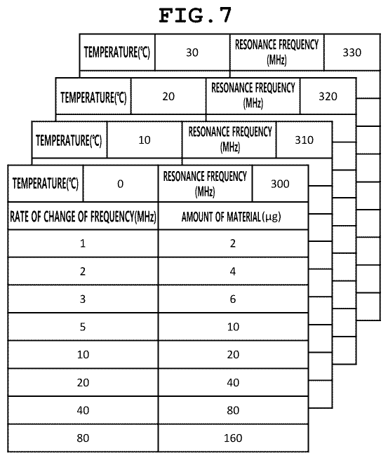

FIG. 7 is a diagram illustrating a reference table based on an embodiment. FIG. 3 will also be used for description.

The reference table may be used to correct changes in the characteristics of the sensor 120 depending on the surrounding environment and accurately measure the amount of the target material. The reference table may be stored in the storage unit 273. The reference table may include values determined by experimentation. The reference table may differ depending on the target material. A plurality of reference tables may be included in one sensor 120 depending on the temperature or humidity. The reference table shown in FIG. 7 illustrates a plurality of reference tables according to temperatures for the sensor 120 that measures a specific target material. Similar to the reference table shown in FIG. 7, a reference table related to humidity may also be used, and a reference table reflecting both temperature and humidity may also be used.

In the multi-channel drug detection method based on an embodiment, the initial state recognition operation S30 may include the operation S24 of determining, as the first resonance frequency, the resonance frequency determined in the resonance frequency search operation S20 performed before inserting the sample, and the operation S52 of selecting a reference table corresponding to the first resonance frequency from among a plurality of reference tables stored in advance, and the plurality of reference tables may include a first resonance frequency value of the sensor 120 according to temperature or humidity, and may include the amount of target material matched according to the amount of change in resonance frequency at a specific temperature or humidity.

The operation S52 of selecting a reference table may be performed after the operation S24 of determining the first resonance frequency. In a state where no sample is inserted, changes in the characteristics of the sensor 120 due to temperature or humidity may be reflected in the first resonance frequency. The first resonance frequency determined in the first resonance frequency search operation S20 includes changes in the characteristics of the sensor 120 depending on temperature or humidity. Accordingly, the processor 260 may further perform the operation S52 of selecting a reference table matching the first resonance frequency, and calculate the amount of the target material using the selected reference table.

In the operation of determining the amount of the target material, the processor 260 may determine the amount of the target material according to the amount of change in resonance frequency by referring to the reference table including the amount of the target material matched according to the amount of change in resonance frequency at a specific temperature or humidity. The processor 260 determines the first resonance frequency in the first resonance frequency determination operation S31 and then selects a reference table corresponding to the first resonance frequency. When the selected reference table is the first resonance frequency, the amount of target material is matched according to the change in resonance frequency. The selected reference table may include the amount of change in resonance frequency and the amount of target material that are matched according to a test performed at a temperature when the sensor 120 has a first resonance frequency. The processor 260 may determine the second resonance frequency and then calculate the amount of the target material according to the change in the resonance frequency by referring to the selected reference table. The second resonance frequency also reflects the influence of temperature or humidity. When determining the amount of the target material, the amount is determined with reference to the reference table, and thus the amount of the target material for which the influence of the surrounding environment has been automatically corrected may be calculated.

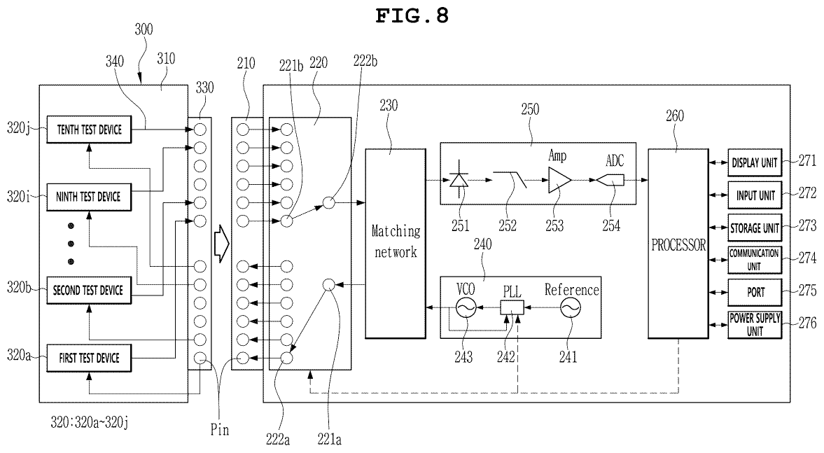

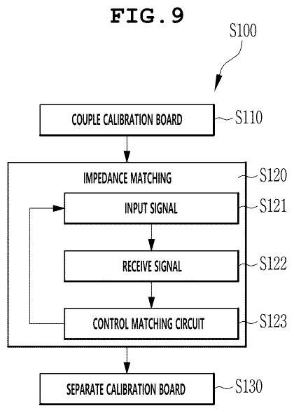

FIG. 8 is a diagram illustrating the calibration board 300 and the main body 200 based on an embodiment. FIG. 9 is a diagram describing a calibration operation based on an embodiment.

The multi-channel drug detection device 1 based on an embodiment may include a third connector 330 connected to the second connector 210, and may further include the calibration board 300 coupled to the main body 200 to perform calibration before connecting the sensor board 100 having a different operating frequency.

Calibration is a process of matching the impedance of the main body 200 to 50 Ohm according to the operating frequency of the sensor board 100 when an operating frequency band of the sensor board 100 changes. In the case of using the sensor board 100 having the same operating frequency, when calibration is initially performed, there is no need to perform calibration again when replacing the sensor board 100 later. However, in order to use a sensor board 100 having a different operating frequency from that of the previously used sensor board 100, it is necessary to perform calibration. A reason therefor is that, when the operating frequency of the sensor board 100 is different, the impedance matched to 50 Ohm becomes different.

The calibration board 300 may include a test board 310, a test device 320, a third connector 330, and a test line 340. The test board 310 may be the same as the sensor substrate 110 of the sensor board 100. The test device 320 is a device that may output the input electrical signal without change. The test device 320 may include a capacitor. A plurality of test devices 320 may be connected through the third connector 330 and the test line 340. The test line 340 may be the same as the sensor line 140 of the sensor board 100. The test line 340 may be in a state impedance-matched to a predetermined value (50 Ohm). Matching may be performed so that the first test device 320a corresponds to the first sensor 120a and the second test device 320b corresponds to the second sensor 120b and, similarly, the ninth test device 320i corresponds to the ninth sensor 120i and the tenth test device 320j corresponds to the tenth sensor 120j.

The multi-channel drug detection method based on an embodiment may further include a calibration operation S100. The calibration operation S100 may further include an operation S110 of coupling the calibration board 300 to the main body 200 before combining the sensor board 100 having an operating frequency band different from the existing one, an impedance matching operation S120 of inputting an electrical signal for each of the plurality of test devices 320 of the calibration board 300, receiving an electrical signal output by the test device 320, and controlling the matching circuit 230 so that the impedance of the path through which the electrical signal is transmitted is matched to a predetermined value, and an operation S130 of separating the calibration board 300 from the main body 200.

When the calibration operation S100 is performed, the impedance of the path through which the electrical signal is transmitted from the oscillator 240 of the main body 200 to the second connector 210 and from the second connector 210 to the receiver 250 may be matched to 50 Ohm. Since the sensor board 100 is manufactured with the impedance of the sensor line 140 matched to 50 Ohm according to an operating frequency range during a manufacturing process, calibration is performed to match the impedance of the signal transmission path of the main body 200. The calibration operation S100 may be performed before the detection operation S1 using the sensor board 100 having a different operating frequency band.

The impedance matching operation S120 may include an operation S121 in which the oscillator 240 inputs an electrical signal of a set frequency to the plurality of sensors 120, an operation S122 in which the receiver 250 receives electrical signals output from the plurality of sensors 120, digitizes the electrical signals, and provides the electrical signals to the processor 260, and an operation S123 in which the processor 260 controls the matching circuit 230 so that the impedance of the path through which the electrical signal is transmitted from the main body 200 is matched to a predetermined value based on the magnitude of the signal received from the receiver 250.

The operation S121 of inputting an electrical signal and the operation S122 of receiving an electrical signal are similar to content described in the resonance frequency search operation S20, and thus overlapping descriptions will be omitted. In the operation S121 of inputting an electrical signal, the processor 260 may control the oscillator 240 so that an electrical signal of a predetermined frequency is generated, and control the switching unit 220 so that a specific test device 320 is connected. In the operation S122 of receiving an electrical signal, the receiver 250 may receive an electrical signal output from the test device 320 and provide the electrical signal to the processor 260. Based on the received electrical signal, the processor 260 may perform the operation S123 of controlling the matching circuit 230 so that the impedance of each of the path through which the electrical signal is transmitted from the oscillator 240 to the second connector 210 and the path through which the electrical signal is transmitted from the second connector 210 to the receiver 250 is matched to 50 Ohm. In order to verify whether the impedance becomes 50 Ohm as a result of performing the operation S123 of controlling the matching circuit 230, the operation S121 of inputting an electrical signal and the operation S122 of receiving an electrical signal may be performed again, and whether the impedance becomes 50 Ohm may be verified, and the operation S123 of controlling the matching circuit 230 may be repeatedly performed.

When the impedance is matched to 50 Ohm, the operation S130 of separating the calibration board 300 may be performed. It is possible to perform a process of measuring the target material by separating the calibration board 300 and connecting the sensor board 100.

When the calibration operation S100 is performed through this process, the impedance of the main body 200 may be matched to 50 Ohm to use the sensor board 100 having a different operating frequency range. In order to detect other chemicals, gases, and biological substances such as DNA in addition to drugs, sensors 120 having different operating frequency ranges may be used. For example, when the operating frequency of the sensor board 100 for detecting drugs is between 100 MHz and 300 MHz, and the operating frequency of the sensor board 100 for detecting gas is between 200 kHz and 400 kHz, it is necessary to perform the calibration operation S100 before replacing the sensor board 100. The main body 200 based on an embodiment has versatility since it is possible to use the sensor board 100 in various operating frequency ranges through calibration.

In addition, as a result of using the pin array-type connector to reduce the size and cost of the sensor board 100 in the multi-channel drug detection device 1 based on an embodiment, it is possible to solve, using the matching circuit 230, a problem of impedance mismatching due to loss of an electrical signal, etc. that may occur when the pin-array type connector transmits a high-frequency electrical signal.

According to the present disclosure, by replacing the reference sensor of the sensor board with an oscillator, the sensor board may be miniaturized and the types of chemicals that can be detected may be increased by one.

According to the present disclosure, the connector connecting the sensor board and the main body is miniaturized, so that various samples may be tested in the field using a plurality of sensor boards.

According to the present disclosure, since the changed resonance frequency of the acoustic wave sensor is tracked using the weighted Kalman filter, a tracking time may be significantly reduced, thereby reducing a sample testing time.

The present disclosure has been described in detail above through specific embodiments. The embodiments are used to specifically describe the present disclosure, and the present disclosure is not limited thereto. It will be clear that modifications and improvements may be made by those skilled in the art within the technical spirit of the present disclosure.

All simple modifications or changes to the present disclosure fall within the scope of the present disclosure, and the specific scope of protection of the present disclosure will be made clear by the appended claims.

What is claimed is:

1. A multi-channel drug detection device comprising:
a sensor board comprising a plurality of sensors into each of which a sample is inserted; and
a main body connected to the sensor board, the main body configured to analyze an electrical signal provided from the sensor board and determine whether a target material is preset in the sample.

2. The multi-channel drug detection device according to claim 1, wherein:
the sensor board comprises a first connector electrically connected to the plurality of sensors,
the main body comprises a second connector connected to the first connector, and
the first connector and the second connector are pin array-type connectors.

3. The multi-channel drug detection device according to claim 2, further comprising a calibration board which comprises a third connector connected to the second connector and coupled to the main body to perform calibration before connecting a sensor board having a different operating frequency.

4. The multi-channel drug detection device according to claim 3, wherein:
the main body comprises:
a switching unit connected to the second connector and configured to select one of the plurality of sensors of the sensor board; and
a matching circuit connected to the switching unit and configured to match impedance of a path through which an electrical signal is transmitted to one of the sensors of the sensor board to a reference value, and
calibration is configured to be performed while the calibration board is connected to the second connector.

5. The multi-channel drug detection device according to claim 1, wherein:
the main body comprises:
an oscillator configured to provide electrical signals of predetermined frequencies to the plurality of sensors of the sensor board;
a receiver configured to generate a signal representing a magnitude of an electrical signal obtained from the sensor; and
a processor configured to control the oscillator so that the predetermined frequencies are generated and to determine whether a target material is present in the sample based on a signal obtained from the receiver,
the processor configured to determine an amount of the target material with reference to a reference table to correct a change in a resonance frequency of the sensors according to temperature and humidity.

6. The multi-channel drug detection device according to claim 5, wherein the processor is configured to determine a weight according to a speed of the resonance frequency that changes immediately after the sample is inserted into the sensor board, and predict a frequency range within which the resonance frequency is to be searched for using a Kalman filter using the weight.

7. A multi-channel drug detection method comprising:
inputting an electrical signal of a frequency included in a search range set in sensors of a sensor board, comparing magnitudes of electrical signals output by the sensors, and determining a frequency having largest magnitude as a resonance frequency, the inputting, comparing, and determining being repeatedly performed at set times;
determining, as a first resonance frequency, a resonance frequency determined in the inputting, comparing, and determining performed before a sample is inserted;
inserting the sample into the sensor board;
determining a second resonance frequency by repeating the inputting, comparing, and determining while moving the search range to search for a resonance frequency changed by a target material included in the sample; and
determining presence of a target material based on a difference between the first resonance frequency and the second resonance frequency.

8. The multi-channel drug detection method according to claim 7, wherein the inputting, comparing, and determining comprises:
setting, by a processor, a search range centered on a resonance frequency in design of the sensor board;
sequentially inputting, by an oscillator, a plurality of electrical signals to the sensors within the search range;
receiving, by a receiver, electrical signals output by the sensors, digitizing the received electrical signals, and providing magnitudes of the received electrical signals to the processor; and
determining, by the processor, whether a peak is present when a frequency output by the oscillator and magnitude of an electrical signal received by the receiver are depicted on a two-dimensional plane, and determining a frequency corresponding to the peak as a resonance frequency.

9. The multi-channel drug detection method according to claim 7, wherein determining the second resonance frequency comprises:
detecting a resonance frequency changing immediately after the sample is introduced and comparing the resonance frequency with an existing resonance frequency to calculate a rate of change of the resonance frequency;
selecting a weight proportional to the rate of change of the resonance frequency;
predicting a frequency at which a resonance frequency is predicted to be present by inputting a frequency of a current electrical signal, a magnitude of an electrical signal provided from the receiver, and the weight to a Kalman filter, and moving a search range, within which search is to be performed in the inputting, comparing, and determining, centered on the predicted frequency; and
performing the inputting, comparing, and determining according to the moved search range, performing the moving the search range again when the resonance frequency is not determined since there is no peak, and determining a resonance frequency as a second resonance frequency when the resonance frequency is determined.

10. The multi-channel drug detection method according to claim 7, wherein:
determining as the first resonance frequency comprises:
determining, as a first resonance frequency, a resonance frequency determined in the inputting, comparing, and determining performed before inserting the sample; and
selecting a reference table corresponding to the first resonance frequency from among a plurality of reference tables stored in advance, and
the plurality of reference tables comprise a first resonance frequency value of a sensor according to temperature and humidity, and comprise an amount of a target material matched according to an amount of change in resonance frequency at a specific temperature and humidity.

11. The multi-channel drug detection method according to claim 10, further comprising determining, by the processor, an amount of a target material according to an amount of change in resonance frequency with reference to a reference table comprising an amount of a target material matched according to an amount of change in resonance frequency at a specific temperature and humidity.

12. The multi-channel drug detection method according to claim 7, further comprising:
coupling a calibration board to a main body before coupling to another sensor board having an operating frequency band different from an existing operating frequency band;
inputting an electrical signal for each of a plurality of test devices of the calibration board, receiving electrical signals output by the test devices, and controlling a matching circuit so that impedance of a path through which the electrical signal is transmitted is matched to a predetermined value; and
separating the calibration board from the main body.

13. The multi-channel drug detection method according to claim 12, wherein the inputting, receiving, and controlling comprises:
inputting, by the oscillator, electrical signals of frequencies included in an operating frequency band of the sensor board to the plurality of sensors;
receiving, by the receiver, electrical signals output from the plurality of sensors, digitizing the electrical signals, and providing the electrical signals to the processor; and
controlling, by the processor, the matching circuit so that impedance of a path through which an electrical signal is transmitted in the main body is matched to a predetermined value based on magnitude of a signal provided from the receiver.
